(19) United States
(12) Patent Application Publication
(10) Pub. No.: US 2011/0077230 A1
(43) Pub. Date: Mar. 31, 2011

(54) COPPER ORGANOCOMPLEXES, USE THEREOF AS ANTITUMOR MEANS AND FOR PROTECTING HEALTHY TISSUE FROM IONIZING RADIATION

(75) Inventors: Valeriy Tatarskiy, Potsdam (DE); Aram Prokop, Berlin (DE)

(73) Assignees: VALERIY TATARSKIY, Potsdam (DE); CHARITE - UNIVERSITATSMEDIZIN BERLIN, Berlin (DE)

(21) Appl. No.: 12/918,131

(22) PCT Filed: Feb. 20, 2009
(86) PCT No.: PCT/EP2009/052073

§ 371 Date: Nov. 10, 2010

(30) Foreign Application Priority Data
Feb. 22, 2008 (EP) 08151840.9

(51) Int. Cl. A61K 31/555; C07F 1/08; A61K 31/30; A61P 35/00

(52) U.S. Cl. 514/184; 556/110; 514/499; 544/225

(57) ABSTRACT

The present invention relates to copper-organic complexes and pharmaceutical compositions containing the same. They can be used especially in the treatment of diseases caused by hyperproliferative cells and, in addition, protect healthy tissue from ionizing radiation. They are prepared by reacting a copper(II) acylate with an organic compound selected from 4-[bis(2-chloroethyl)amino]-D,L-phenyl-alanine (sarcolysine), the hydrochloride thereof, N-(2-furanidil)-5-fluorouracil (tegafur) and aminocarbonylaziridine (leacadin) at acidic to neutral pH in a chloroform/methanol mixture.

The present invention relates to copper-organic complexes and pharmaceutical compositions containing the same. They can be used especially in the treatment of diseases caused by hyperproliferative cells and, in addition, protect healthy tissue from ionizing radiation. They are prepared by reacting a copper(II) acylate with an organic compound selected from 4-[bis(2-chloroethyl)amino]-D,L-phenyl-alanine (sarcolysine), the hydrochloride thereof, N-(2-furanidil)-5-fluorouracil (tegafur) and aminocarbonylaziridine (leacadin) at acidic to neutral pH in a chloroform/methanol mixture.

Hyperproliferative cells are the cause of various diseases, particularly clinical patterns summarized under the term “cancer”, with frequently fatal outcome. Excessive proliferation results in an imbalance between new growth of tissue and controlled death of cells in the tissue structure. The natural homeostasis is disturbed. This delicate balance between tissue formation and degradation is regulated by the process of apoptosis. Apoptosis refers to programmed cell death which can be performed by any cell. As a response to specific signals, intracellular processes are triggered that result in self-disintegration of the cell without causing inflammatory processes. In this way, excessive proliferation of cells and tissues is prevented.

In the treatment of diseases caused by excessively proliferating cells, attempts are being made in clinical practice—apart from surgical removal of localizable tumors—to destroy the tumor cells and reestablish the balance by means of cytotoxic measures such as chemotherapy, radiation therapy and hyperthermia. Time and again, however, it has been found that part of the malignant tumors develop radio- or chemoresistance at a quite early stage or are even primarily refractory to therapy. Primary tumor and metastases are sometimes quite different in their responsive behavior to a particular therapy. As a result of such resistance, a large number of cancer therapies have remained unsatisfactory. In particular, this applies to the treatment of relapse in childhood acute lymphoblastic leukemia (ALL). About 60% of the children suffering from ALL relapse die despite aggressive cytotoxic therapy. Other tumor diseases difficult to treat are, for example, mammary, bronchial, thyroid and prostate carcinomas as well as melanoma, neuroblastoma, medulloblastoma, astrocytoma and glioblastoma. Similarly, benign conditions resulting from hyperproliferative cells are being treated with cytostatic medicaments still requiring substantial improvement in their therapeutic potency.

WO 03/004014 A1 has already described monocrystalline diketone copper complexes of melphalan, tegafur and leacadin, which can be used as antitumor agents. Also, melphalan, tegafur and leacadin themselves are known as cytostatic agents.

However, there is a strong interest in and great need for the development of other substances capable of improving the remedial success and chances of survival. The invention was therefore based on the object of finding compounds which have highly selective effectiveness against excessively proliferating, drug-resistant cells and are suitable for the treatment of tumor diseases and leukemias without doing excessive damage to healthy cells. Another object of the invention was to provide compounds which protect healthy tissue during medical use of ionizing radiation on humans to cure diseases or delay the progression thereof.

The object of the invention is accomplished by means of new copper-organic complexes which can be obtained by reacting a copper(II) acylate with an organic compound at acidic to neutral pH in a chloroform/methanol mixture. According to the invention, the organic compound is selected from 4-[bis(2-chloroethyl)amino]-D,L-phenylalanine (D,L-melphalan, sarcolysine), the hydrochloride thereof, N-(2-furanidil)-5-fluorouracil (tegafur) and aminocarbonylaziridine (2-carbamoylaziridine, leacadin). In a preferred fashion, copper(II) acetate or copper(II) propionate is used as copper acylate.

It was found that the copper-organic complexes according to the invention are highly selective against excessively proliferating, drug-resistant cells and suitable for use in the treatment of tumor diseases and leukemias. Surprisingly, they are also capable of protecting healthy tissue from radiation damage, e.g. during radiation treatment following tumor resection or in tumor treatment by irradiation, and can therefore be used in radiotherapy. Furthermore, it was found that the copper-organic complexes of the invention exhibit a synergistic effect in the treatment of cancer when combined with per se known cytostatic agents. Moreover, the copper-organic complexes according to the invention exhibit a pronounced antioxidative activity and therefore can also be used as antioxidants in the treatment of inflammatory diseases which may be associated with tumor diseases, for example.

The copper complexes according to the invention are preferably prepared by mixing and heating solutions of the starting materials. The complex precipitates upon cooling and is purified by repeated washing with a chloroform/methanol mixture as solvent and subsequently dried. For successful reaction, a narrow pH range in an acidic to neutral environment must be maintained. The complexes are microcrystalline compounds wherein the copper is in a tetragonal structure and the organic compound is bound to the copper via oxygen and/or nitrogen atoms.

The method according to the invention is preferably characterized in that the copper(II) acylate and the organic compound selected from 4-[bis(2-chloroethyl)amino]-D,L-phenylalanine (sarcolysine), the hydrochloride thereof, N-(2-furanidil)-5-fluorouracil (tegafur) and aminocarbonylaziridine (leacadin) are each dissolved in a chloroform/methanol mixture. After combining, the solution is acidified, if necessary, and heated to a preferred temperature of 50° C., optionally to boiling. The precipitate resulting after cooling is washed, if necessary, and dried, preferably in air. The chloroform/methanol mixture is preferably used at a ratio of from 1:1 to 3:1.

According to the invention, the following copper-organic complexes are preferably provided:
a) Copper-sarcolysine hydrochloride complex (complex A) having a melting point of 147° C., which complex comprises Cu: 15.0%, C, 37.3%, H, 4.43%, Cl: 24.4%, N 6.74% and oxygen and is prepared e.g. by reacting sarcolysine hydrochloride and copper(II) acetate at a pH of about 2 to about 3.
b) Copper-sarcolysine complex (complex B) having a melting point of 177° C., which complex comprises Cu: 9.2% C, 45.3% H 5.4% Cl: 20.6% N: 8.1% and oxygen and is prepared e.g. by reacting sarcolysine and copper(II) acetate at a pH of about 6 to about 7.
c) Copper-tegafur complex (complex C) having a melting point of 127° C., which complex comprises 26.8% Cu, 18.16% carbon, 3.08% hydrogen, 25.3% chlorine, 3.39% nitrogen as well as oxygen and fluorine and is prepared e.g. by reacting tegafur and copper(II) acetate at a pH value of about 1.

The deviations of the quoted analytical values for carbon, nitrogen and chlorine are less than 3% on average, and the deviations of the quoted analytical values for hydrogen and copper are up to 4.5% on average.

The invention is also directed to a pharmaceutical composition comprising at least one new copper-organic complex and optionally pharmaceutical adjuvants and/or excipients.

The pharmaceutical composition is prepared according to per se known methods, the complex compound of the invention preferably being provided in combination with suitable pharmaceutical excipients. The content of active substance in this composition is normally from 0.1 to 99.5 wt. %, preferably from 0.5 to 95 wt. % of the total mixture.

The pharmaceutical composition according to the invention can be provided in different forms of administration. As a rule, it consists of at least one complex compound of the invention and non-toxic, pharmaceutically tolerable excipients which are used as admixture or diluent, e.g. in solid, semi-solid or liquid form, or as a coating agent, e.g. in the form of a capsule, a tablet coating, a bag or other container for the therapeutically active component. An excipient can be used e.g. as a mediator for drug absorption in the body, as a formulation aid, sweetener, taste corrigent, dye or preservative.

Administration of the pharmaceutical composition of the invention is mainly peroral, intraperitoneal, intratracheal, intraabdominal, intravenous, transdermal or intramuscular or in an administration form using microenemas. Pulmonary administration is also possible.

Sterile injectable aqueous solutions, isotonic saline solutions or other solutions are used for parenteral application of the pharmaceutical composition.

Aqueous suspensions may contain suspending agents, e.g. sodium carboxymethylcellulose, methylcellulose, hydroxypropylcellulose, sodium alginate, polyvinylpyrrolidone, tragacanth or gum arabic, dispersing and wetting agents, e.g. polyoxyethylene stearate, heptadecaethyleneoxycetanol, polyoxyethylenesorbitol monooleate or lecithin; preservatives, e.g. methyl or propyl hydroxybenzoate; flavoring agents, sweeteners, e.g. saccharose, sodium cyclamate, glucose, invert sugar syrup.

Oily suspensions may contain, for example, peanut, olive, sesame, coconut or paraffin oil, and thickening agents such as beeswax, hard paraffin or cetyl alcohol, and also sweeteners, flavoring agents, and antioxidants.

Water-dispersible powders and granules may contain the complex compound according to the invention in a mixture with dispersants, wetting agents and suspending agents, e.g. those mentioned above, optionally together with sweeteners, flavoring agents and dyes.

Emulsions may contain, for example, peanut, olive or paraffin oil in addition to emulsifiers such as tragacanth, gum arabic, polyoxyethylenesorbitan monooleate, as well as flavoring agents and sweeteners.

Aqueous solutions may contain preservatives, e.g. methyl or propyl hydroxybenzoate; thickening agents, flavoring agents, sweeteners, e.g. saccharose, sodium cyclamate, glucose, invert sugar syrup, as well as dyes.

Olive oil, DMSO, Tween 60 or 80, or physiological saline are preferably used as diluents and solvents.

For example, tablets, coated tablets, capsules, e.g. of gelatin, dispersible powders, granulates, aqueous and oily suspensions, emulsions, solutions or syrups can be used for oral application. Tablets may contain inert fillers, e.g. starches, lactose, microcrystalline cellulose, glucose, calcium carbonate or sodium chloride; binders, e.g. starches PEGs, PVP, gelatin, cellulose derivatives, alginates or gum arabic; lubricants, e.g. magnesium stearate, glycerol monostearate, stearic acid, silicone oils or talc; disintegrants, taste corrigents or dyes.

For oral application, the pharmaceutical composition is preferably used in the form of a fine dispersion in oil, or as an intravenous injection or infusion solution, or in the form of tablets. Surprisingly, the copper-organic complexes develop their full effect even in oral administration, which means considerable advantages in handling and alleviation for the patient.

Advantageously, the pharmaceutical preparation of the active substance is in the form of unit doses adjusted to the desired administration. For example, a unit dose can be a tablet, capsule, suppository or an appropriate volume quantity of a powder, granulate, solution, emulsion or suspension or dispersion.

In general, the complexes according to the invention are administered in a daily dose of from 0.01 to 10 mg/kg body weight, optionally in the form of multiple single doses to achieve the desired result. A single dose contains the complex(es) in amounts of from 0.01 to 10 mg/kg body weight.

It was found that the copper complexes of the invention accumulate almost exclusively in tumor tissue and, in addition, are capable of crossing the blood-brain barrier. Consequently, the new copper-organic complexes are preferably used in the treatment of a number of tumor diseases. The compounds of the invention can be used in the treatment of a wide variety of different diseases, especially diseases resulting from highly proliferative cells, e.g. in the treatment of malignant diseases of bone marrow and other hemopoietic organs, leukemias, solid tumors, epithelial tumors, sarcomas and malignant and semimalignant diseases of the skin. The complexes according to the invention strongly induce apoptosis of cancer cells even if these cells are resistant to conventional therapeutic agents. In malignant cells, they induce expression of the p53 protein and opening of the cyclosporin A pores.

For example, in leukemia cells resistant to daunorubicin and doxorubicin the copper-organic complexes according to the invention increase the induction of apoptosis by two to six times compared to conventional cytotoxic agents (see Example 5 and FIG. 12).

Another preferred use is in radiation therapy, mainly to protect healthy tissue from ionizing radiation. Radiation therapy (also referred to as radiology, radiotherapy, radiooncology) is a medical field dealing with the medical application of ionizing radiation on humans and animals to cure diseases or retard the progression thereof. The purpose of radiation therapy is direct destruction of tumor cells. The field of radiation therapy also comprises medicamentous and physical methods for radiosensitization and amplifying the radiation effect on a tumor (chemoradiotherapy), taking account of protective measures for the healthy tissue. Gamma rays, X-ray bremsstrahlung and electrons are mainly used as ionizing, high-energy radiation. Also, instruments for the treatment with neutrons, protons and heavy ions have been constructed in recent years. According to the invention, the copper-organic complexes are used to protect healthy tissue, optionally in combination with well-known radiotherapeutic measures and means. To this end, the complexes of the invention are used in accordance with the above-described administration forms and doses.

In another embodiment of the invention, the copper-organic complexes can be used in combination with at least one additional cytostatic agent (tumor therapeutic agent).

More specifically, the copper-organic complex compounds according to the invention are suitable for the treatment of the following tumor diseases, to which end they are used alone or in combination with radiotherapeutic measures/means and/or in combination with conventional tumor agents: intestinal carcinoma, brain tumor, eye tumor, pancreatic carcinoma, bladder carcinoma, lung cancer, breast cancer, ovarian tumor, cervical tumor, skin cancer, testicular cancer, kidney tumor, germ cell tumor, liver cancer, leukemia, malignant lymphoma, nerve tumor, neuroblastoma, prostate cancer, soft tissue tumor, esophageal cancer as well as carcinomas with unknown primary tumor.

In a preferred fashion, a patient having a tumor disease is administered with the pharmaceutical composition in an amount sufficient to achieve treatment of the respective tumor. The amount of pharmaceutical composition to be administered depends on a number of factors, such as selection of the specific copper complex and optionally other, simultaneously administered pharmaceuticals, mode of administration (peroral, infusion, injection, etc.), nature and severity of the tumor disease, as well as age, weight and general condition of the patient. The amount can easily be determined by a person skilled in the field of tumor diseases, taking into account the above-mentioned factors. As a rule, the copper complexes are preferably administered in the course of treatment in single doses ranging from 0.01 mg/kg body weight of the patient up to 10 mg/kg body weight of the patient and more preferably from 0.5 to 5 mg/kg, especially preferably at dosages of from 0.5 to 1.5 mg/kg body weight of the patient.

The term “tumor” as used herein encompasses any local increase in tissue volume, as well as cells in which normal growth regulation no longer applies and uncontrolled cell division takes place, that is, neoformation of tissue (e.g. tumescence, blastoma, neoplasia) in the form of spontaneous, diversely uninhibited, autonomous and irreversible excessive growth of autologous tissue, usually associated with differently pronounced loss of specific cell and tissue functions.

The term “treatment of tumors” as used herein comprises at least one of the following features: alleviating the symptoms associated with a tumor disease, lessening the severity of the tumor disease (e.g. reduction of tumor growth), stabilizing the condition of the tumor diseases (e.g. inhibition of tumor growth), preventing further spreading of the tumor diseases (e.g. metastasis), preventing the occurrence or recurrence of a tumor disease, slowing down the progression of the tumor disease (e.g. reduction of tumor growth), or improving the condition of the tumor disease (e.g. reducing the size of the tumor).

The substances of the invention can be unambiguously characterized by way of the preparation method, elemental analysis, melting point, UV/VIS, IR, EPR and NMR spectra. The complexes according to the invention are remarkable for a number of outstanding properties and are therefore clearly superior to conventional chemotherapeutic agents.

The copper complexes according to the invention accumulate almost exclusively in tumor tissue so that the side effects of treatment with the complexes of the invention are comparatively low. Moreover, this opens up the possibility of using the complexes of the invention for detecting malignant cells. Thus, using the complexes according to the invention and conventional methods, e.g. fluorescence microscopy, malignant cells can be detected in tissue when applying the inventive complexes labeled with appropriate fluorescent dyes well-known to those skilled in the art. Similarly, malignant cells can be detected in vitro in extracted tissue using the complexes of the invention.

The complexes according to the invention are capable of crossing the blood-brain barrier and therefore can also be used in the treatment of brain tumors. Furthermore, the use of the copper-organic complexes of the invention effects encapsulation of tumors, so that surgical removal thereof is facilitated.

The invention is also directed to combination preparations comprising the copper complexes of the invention and per se known cytostatic agents such as cytarabine, cladribine, etoposide, fludarabine or idarubicin. Another advantage of the copper-organic complexes of the invention is that, when used in combination with other, conventional cytostatic agents, a significant synergistic increase in effectiveness is found compared to using the single preparations. According to the invention, the copper-organic complexes are therefore used in combination with conventional cytostatic agents. The two components of the combined preparation can be administered either simultaneously or sequentially. The conventional cytotoxic agents are preferably selected from alkylating and crosslinking compounds such as nitrogen mustard derivatives, N-nitroso compounds, ethyleneimine (aziridine) derivatives, platinum complexes, cytostatic antibiotics such as anthracyclines, bleomycin and mitomycin; antimetabolites, such as folic acid antagonists, pyrimidine and purine analogs, antimitotic substances such as vinca alkaloids and taxanes; hormones and hormone antagonists.

Thus, for example, it was found that a combination of copper-sarcolysine hydrochloride (complex A) and an antimetabolite, namely, cytarabine (AraC), results in an increase in effectiveness by 129%. It is noteworthy that this synergistic effect occurs already with a subtherapeutic dose of complex A according to the invention (see Example 7 and FIG. 14).

Consequently, the combination preparation according to the invention is ideally suitable for the treatment of tumor diseases. Owing to the presence of the complexes according to the invention in the preparation, it can also be used to protect healthy tissue during radiotherapy.

The following examples in connection with the figures are intended to explain the invention in more detail.

### LEGENDS TO THE FIGURES

FIG. 1: Diffractometric analysis of complex A

FIG. 2: UV/VIS spectrum of complex A

FIG. 3: IR spectrum of complex A

FIG. 4: EPR spectrum of complex A

FIG. 5: UV/VIS spectrum of complex B

FIG. 6: IR spectrum of complex B

FIG. 7: EPR spectrum of complex B

FIG. 8: UV/VIS spectrum of complex C

FIG. 9: IR spectrum of complex C

FIG. 10: EPR spectrum of complex C

FIG. 11: DNA fragmentation by complex A on BJAB mock

FIG. 12: DNA fragmentation in primary lymphoblasts in comparison between complex A and other cytostatic agents

FIG. 13: Change in mitochondrial membrane potential in BJAB cells after treatment with complex A

FIG. 14: Synergistic effect of complex A with cytarabine in lymphoma cells

FIG. 15: Effect of complex A on human lymphoma cells (BJAB) in mice in peroral administration

FIG. 16: EPR spectra of a Walker carcinosarcoma when treated with complex A

FIG. 17: Influence of complex A and complex C on the NADH state and calcium homeostasis of thymocytes of the Ehrlich ascites carcinoma in rats

FIG. 18: Influence of complex A and complex C on the effect of ionizing radiation using spleens of mice as example

FIG. 19: Antioxidant properties of complex A and complex C

FIG. 20: Induction of cyclosporin A sensor pores by complex A compared to sarcolysine

FIG. 21: Effect of complex A on cell cultures of human laryngeal cancer Hep-2 (nuclear magnetic resonance)

FIG. 22: In vitro effect of complex A on human mammary carcinoma cells (photograph 1: after 4 min incubation; photograph 4: after 18 min incubation; photograph 9: after 43 min incubation)

### EXAMPLES

### Example 1

### Preparation of a copper-organic complex (complex A) of 4-[bis(2-chloroethyl)amino]-D,L-phenylalanine hydrochloride (=sarcolysine hydrochloride)

0.0125 mol of sarcolysine hydrochloride is dissolved in 50 ml of a mixture of 1 part of methanol and 3 parts of chloroform (solution A).

0.01 mol of copper(II) acetate is likewise dissolved in 50 ml of the above solvent mixture (solution B).

Solution B is added to solution A with continuous stirring and heating to boiling temperature. The mixture is held at boiling temperature for another 30 minutes with stirring and subsequently cooled to room temperature. The pH value of the mixture is 2 to 3.

A microcrystalline green precipitate settles within a few hours, which is separated by filtration using a glass frit, washed several times with the above solvent mixture and dried in air at 65 to 70° C.

The resulting copper complex (complex A) is a microcrystalline powder and has a melting point of 147° C. The elemental analysis affords the following composition: Cu: 15.0%, C, 37.3%, H, 4.43%, Cl: 24.4%, N: 6.74%, O: 10.3%.

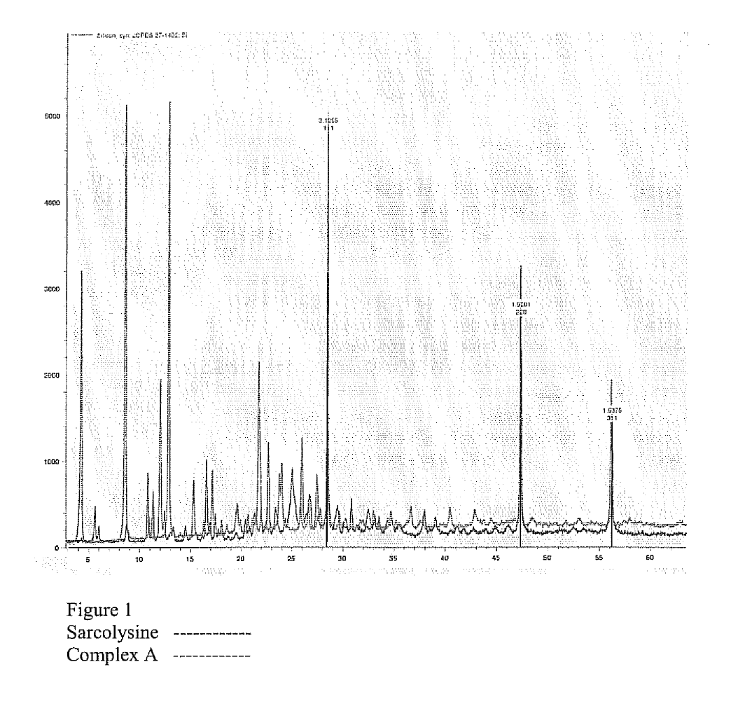

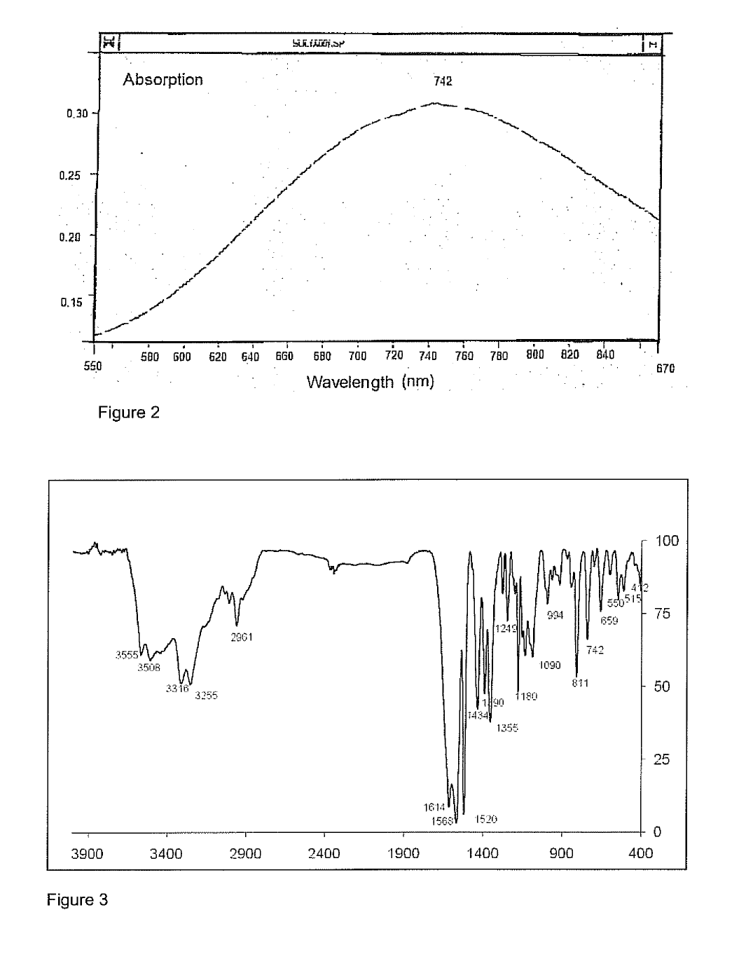

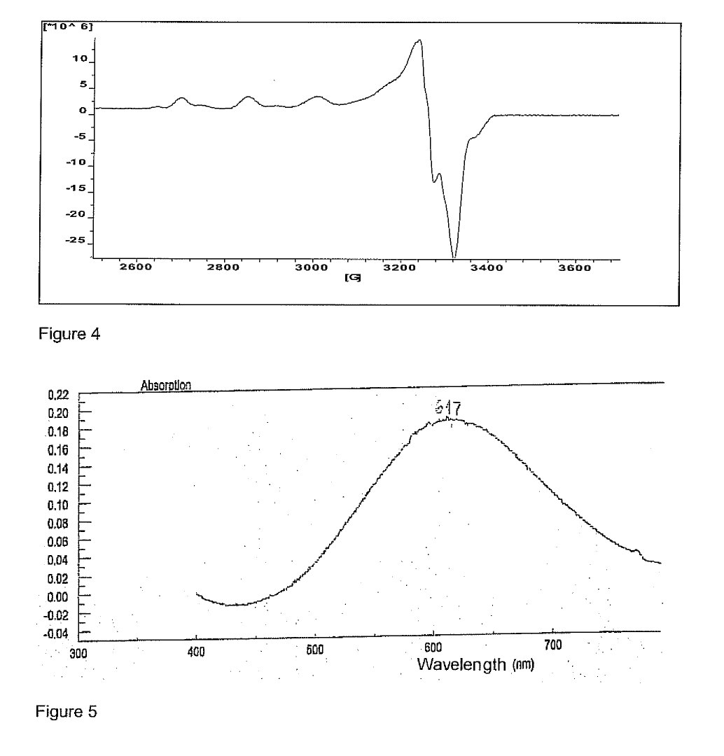

Other physical properties of complex A are shown in FIGS. 1 to 4.

Complex A is insoluble in water and most organic solvents. It dissolves in dimethyl sulfoxide (DMSO) and can be emulsified in vegetable oils and with the aid of suitable emulsifiers (Tween 60 and Tween 80) in aqueous media. It is stable at room temperature even when exposed to oxygen.

### Example 2

### Preparation of a copper-organic complex (complex B) of 4-[bis(2-chloroethyl)amino]-D,L-phenylalanine (=sarcolysine)

0.0125 mol of sarcolysine is dissolved in 50 ml of a mixture of 3 parts of chloroform and 1 part of methanol (solution A).

0.01 mol of copper(II) acetate is dissolved in 50 ml of the above solvent (solution B).

Solution B is added to solution A with continuous stirring and heating. The mixture is held at 50° C. for about 20 minutes with stirring and subsequently cooled to room temperature. The pH value of the mixture is 6 to 7.

A microcrystalline blue precipitate settles within a few hours, which is filtered using a glass frit, washed several times with solvent and dried in air at 65 to 70° C.

The resulting copper complex (complex B) is a microcrystalline powder and has a melting point of 177° C. The elemental analysis affords the following composition: Cu: 9.2%, C, 45.3%, H, 5.4%, Cl: 20.6%, N: 8.1%, O: 11.6%.

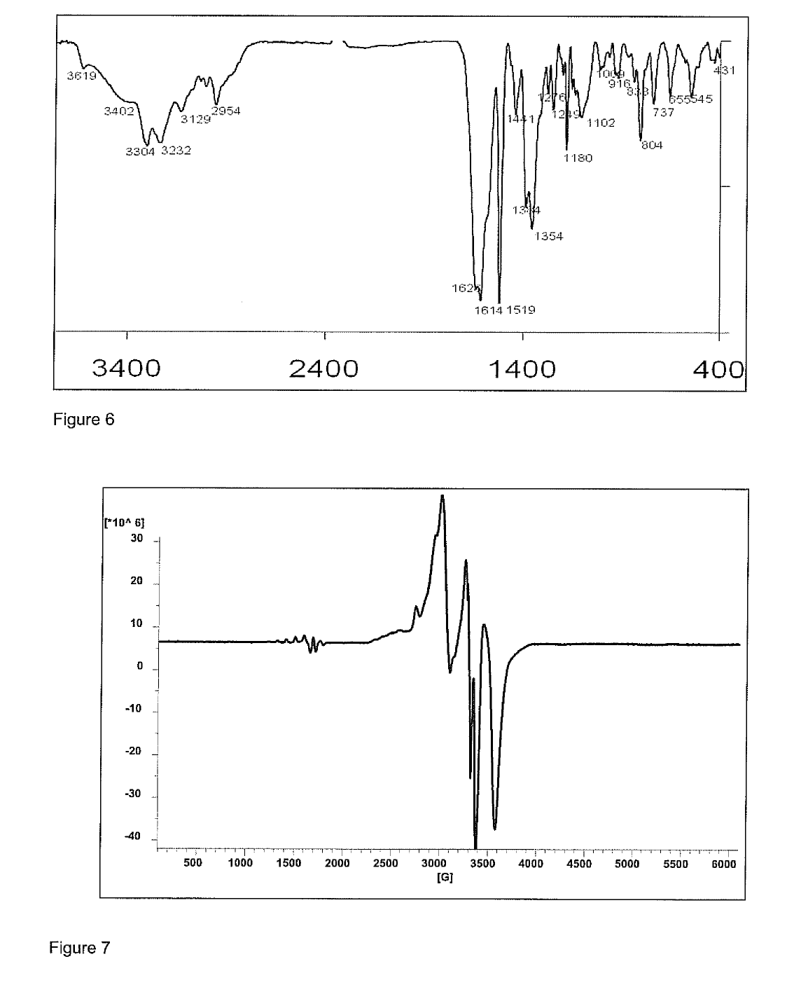

Other physical properties of complex B are shown in FIGS. 5 to 7.

Complex B is insoluble in water and many organic solvents. It dissolves in DMSO and can be dispersed in oil and with the aid of suitable emulsifiers (Tween 60 and Tween 80) in aqueous media.

### Example 3

### Preparation of a copper complex (complex C) of 5-fluoro-1-(tetrahydro-2-furyl)-uracil (=tegafur)

### Variant 1

0.01 mol of copper(II) acetate is dissolved in 50 ml of chloroform (solution A).

0.02 mol of tegafur is dissolved in 50 ml of a mixture of chloroform and methanol at a volume ratio of 1:1 (solution B).

The solutions A and B are heated to boiling in a water bath, combined with continuous stirring and acidified with hydrochloric acid. The mixture is allowed to stand in an open vessel in the dark until the solvents have evaporated. The residue is made free of starting materials by washing with chloroform, and the remaining green crystals are dried in air.

The resulting copper complex (complex C) is a microcrystalline powder and has a melting point of 127° C.

### Variant 2

0.01 mol of copper(II) acetate is dissolved in 50 ml of chloroform/methanol at a volume ratio of 1:1 (solution A).

0.02 mol of tegafur is dissolved in 50 ml of the above solvent (solution B).

The two solutions are heated to boiling, and solution A is added to solution B with continuous stirring. The mixture is held at boiling temperature for another 30 min, while the pH value is held at 1 by metering concentrated HCl. The reacted mixture is evaporated at room temperature, and the dry residue is washed 3 to 4 times with methanol of −18° C. on a glass frit. The wash liquid contains the complex C of the invention, which remains as a residue after evaporating the methanol.

The elemental analysis of complex C affords the following composition: Cu: 26.8%, C, 18.16%, H, 3.08%, Cl: 25.3%, N: 3.39%. Oxygen and fluorine were determined to be 18.2% and 2.09%, respectively. However, the analytical values of fluorine are subject to strong variation and those of oxygen may be distorted towards lower values.

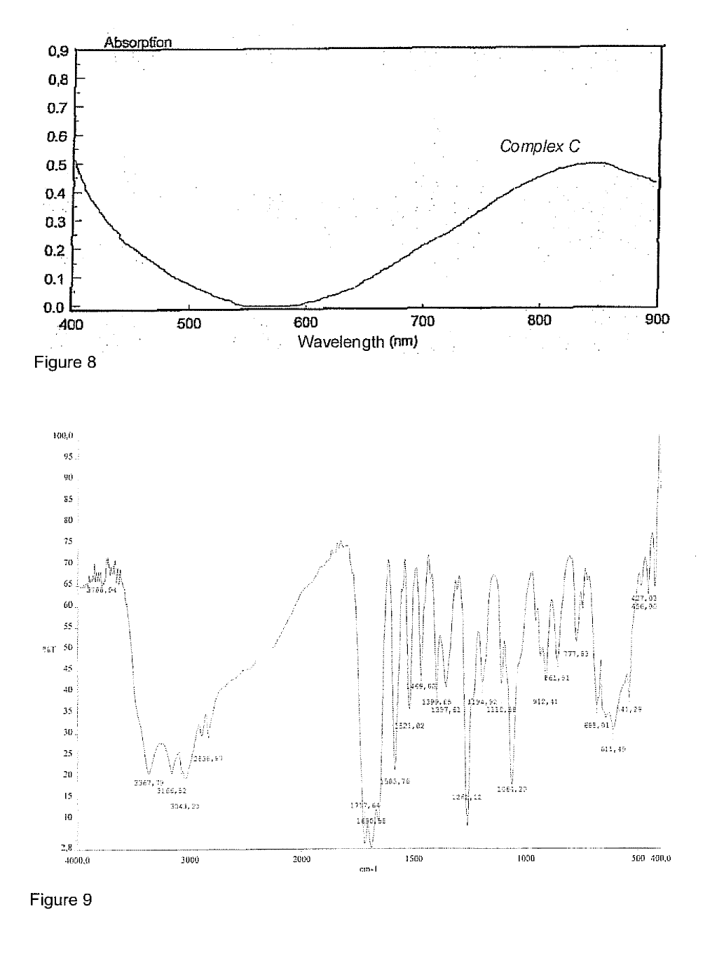

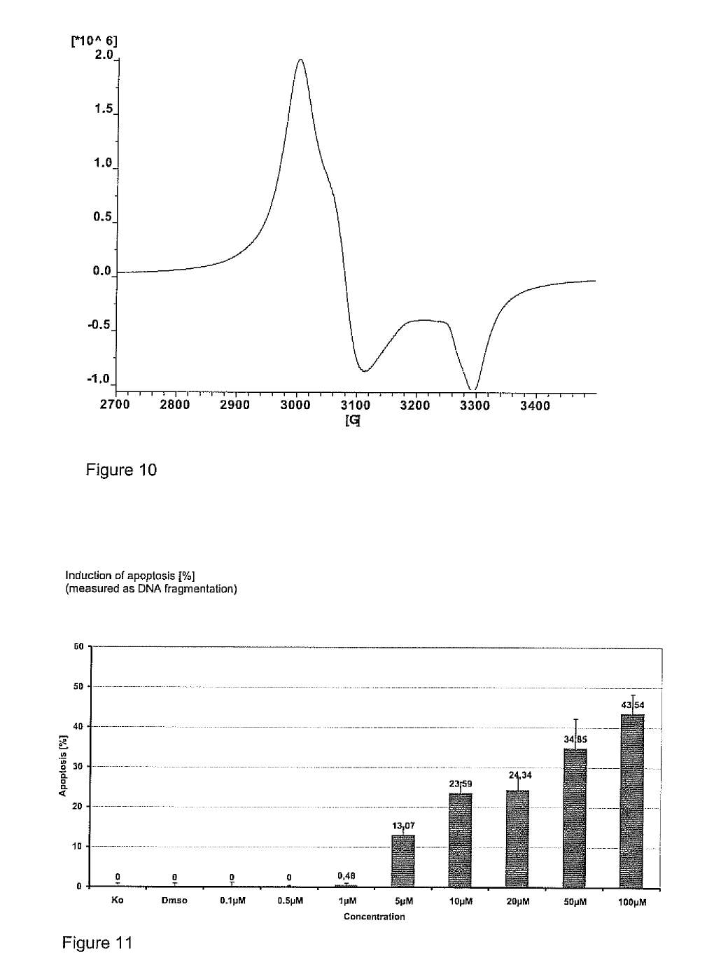

Other physical properties of complex C are shown in FIGS. 8 to 10.

The complex C is readily soluble in water, physiological saline, methanol, ethanol, DMSO and Tween 80. It is insoluble in ether and chloroform. It is stable at room temperature in dry air.

The analytical values specified in Examples 1, 2 and 3 are determined using the following procedure:

The analyses of C, H and N are performed simultaneously using a FlashEA 1112 CHNS/O Automatic Elemental Analyser. The oxygen values are determined separately on the same instrument, in which determination the sample is decomposed at high temperature in the absence of oxygen and the resulting CO is measured. The presence of significant amounts of copper may give rise to distortions in the oxygen value as a result of oxide formation.

For Cu determination, the sample is decomposed with nitric acid and Cu determined in solution by means of emission spectroscopy using an ICP Optima 2100 DV emission spectrometer from Perkin Elmer.

Chlorine is determined by potentiometric titration in a solution obtained by decomposition according to Schrödinger.

For fluorine determination, the substance is likewise subjected to decomposition according to Schrödinger, and the solution is subsequently analyzed by means of anion chromatography using a 761 Compact IC Autosampler ion chromatograph from Metrohm.

Each of the quoted analytical values represents a mean value of a number of measurements. The deviations of the individual values for C, N and Cl are less than 3% of the measured values on average, the deviations for hydrogen and Cu are up to 4.5% of the measured values on average. The analytical values for fluorine show strong variation, and it is difficult to specify a reliable range of variation.

### Example 4

### Influence of Complex A on Induction of Apoptosis

BJAB cells (lymphoma cell line) at a concentration of 1×10⁵/ml were treated with increasing concentrations of complex A dissolved in DMSO and incubated for 72 hours at 37° C. and 5% CO₂. Following propidium iodide staining, the DNA fragments were detected using flow cytometry (FACS analysis). KO (untreated cell suspension) and DMSO were carried along in an equivalent treatment. The result is shown in FIG. 11. As can be seen, complex A causes concentration-dependent induction of apoptosis which reaches a value of 43.5% of the cells at 0.1 mmol of complex A.

### Example 5

### Ex Vivo Effect of Complex A in Primary Lymphoblasts

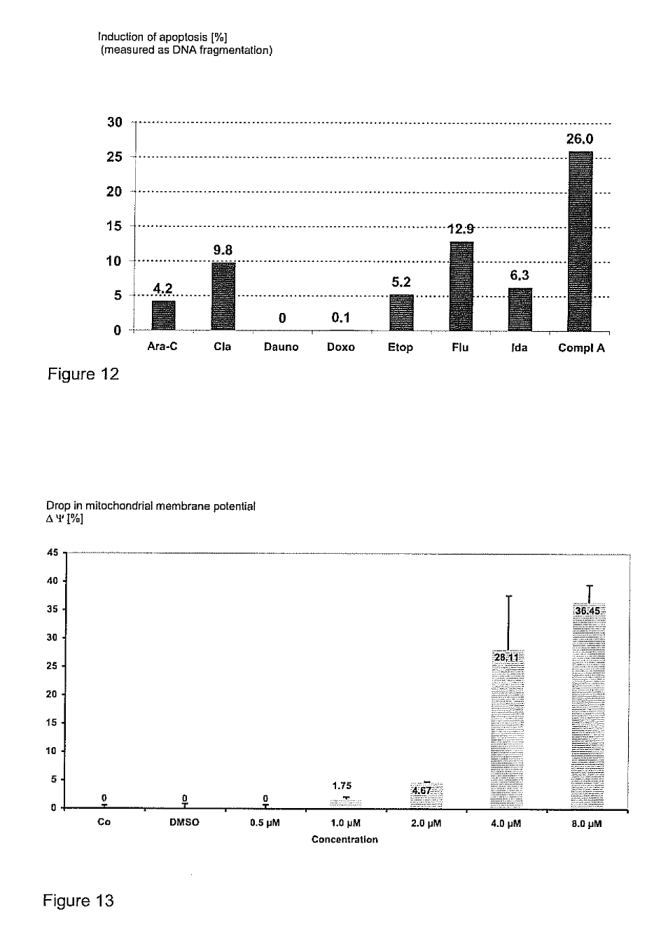

Following extraction from ALL patients, the lymphoblasts were initially isolated and subsequently treated both with commercially available cytostatic agents and complex A. The concentrations were selected so as to be invariably in the LC₅₀ range when using the NALM-6 leukemia cell line. Thereafter, the cells were incubated for 60 hours at 37° C. and 5% CO₂, subsequently stained with propidium iodide and quantified using flow cytometry in an FACS. The results are shown in FIG. 12. As demonstrated impressively, there is induction of apoptosis in leukemia cells resistant to daunorubicin (dauno) and doxorubicin (doxo). Being about 26%, induction of apoptosis by complex A in lymphoblasts is significantly higher compared to the conventional cytostatic agents cytarabine (ARA-C), cladribine (Cla), etoposide (Etop), fludarabine (Flu) and idarubicin (Ida).

### Example 6

### Detection of Mitochondrial Mediation of the Apoptotic Signal Cascade by Complex A

Following treatment of the BJAB cells with different concentrations of complex A, carrying along a zero control and a solvent control, the cells were incubated for 48 hours at a temperature of 37° C. and 5% CO₂. FIG. 13 shows the evaluation of the tests by staining with the mitochondria-specific dye JC-1 and flow cytometric detection of the color change. A concentration-dependent change of the mitochondrial membrane potential induced by complex A is found in more than 30% of the cells.

### Example 7

### Combined Administration of Complex A and Cytarabine Cytostatic Agent

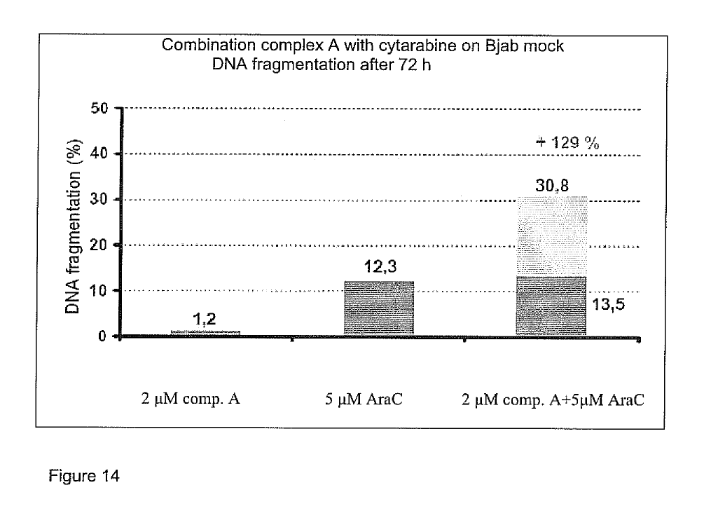

The combined preparation shows marked in vitro synergy effects with conventional cytarabine cytostatic agent in lymphoma cells (BJAB). Incubation was performed for 48 hours at 37° C. and 5% CO₂, carrying along zero and solvent controls. DNA fragmentation is determined by flow cytometry after staining the cells with propidium iodide. FIG. 14 shows the mean value of triple measurements. As can be seen, there is a significant synergy effect of complex A with cytarabine of +129% induction of apoptosis.

### Example 8

### In Vivo Inhibition of Tumor Growth in Mice Using Complex A

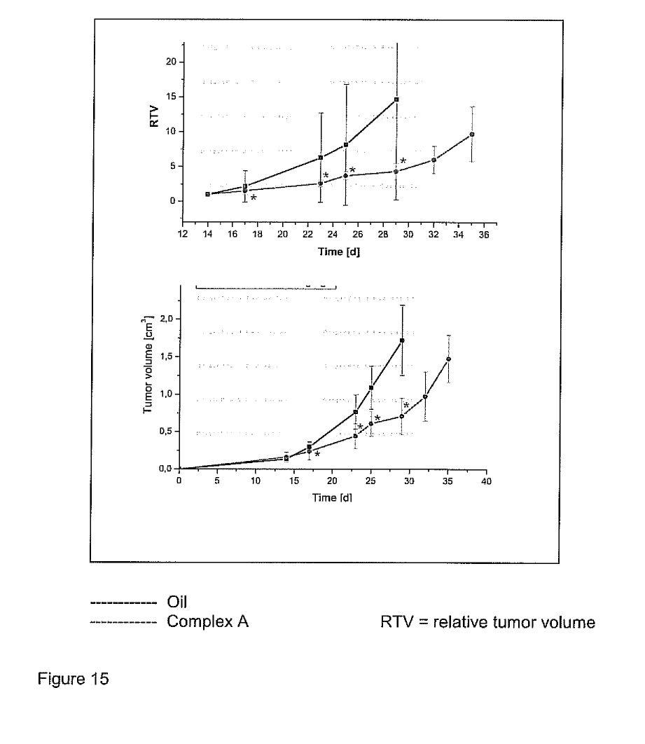

Human lymphoma cells (BJAB) were implanted in six SCID mice. After a tumor with a diameter of 5 mm had formed, the mice were subjected to peroral treatment with complex A at a dose of 10 mg/kg, suspended in olive oil. The control group consisted of 10 animals treated with olive oil only. A significant inhibition of tumor growth was observed following administration of complex A. The result is shown in FIG. 15.

* denotes the significance (p≦0.05) in the Mann-Whitney U-test.

### Example 9

### Effect of Complex A on B 16 Melanoma in Mice

The melanoma B 16 was implanted in 20 mice of the line C 57Bl by subcutaneous injection into the right thigh. The animals were divided into four groups of five mice each. Group I received complex A at a dosage of 7 mg/kg, dissolved in DMSO, which was administered intraabdominally in a 4% suspension in physiological saline. Each administration was performed 3, 5, 7 and 9 days after trans-plantation of the tumor.

Group II was treated as group I, but using a dosage of 5 mg/kg.

Group III was treated as group I, but received 5 mg/kg of melphalan hydrochloride instead of complex A.

Group IV remained untreated.

Table 1 shows the average weight and volume of the tumors in each of the four groups, the inhibition of tumor growth, the average mitotic and apoptotic indices and the mitotic index of the bone marrow.

Table 1 clearly shows the superior antitumor effect of complex A and a significantly lower inhibition of bone marrow production.

TABLE 1

| | Average weight | | Inhibition of | Mitotic index of | Apoptotic | Bone marrow |
|---|---|---|---|---|---|---|
| | and volume (cm³) | | tumor growth | tumor cells MI | index AI | index MI BM |
| Substance | of tumor | | % | ‰ | ‰ | ‰ |
| Group I: | 136.8 | mg | mass percent 89.5 | 1.3. | 4.25 | 3.575 |
| complex A | 0.029 | cm³ | volume percent 98.67 | | | |
| Group II: | 202.25 | mg | mass percent 84.44 | 2.175 | 3.68 | 3.975 |
| complex A | 0.436 | cm³ | volume percent 80.00 | | | |
| Group III: | 482.6 | mg | mass percent 62.87 | 2.43 | 3.07 | 1.88 |
| sarcolysine | 1.4 | cm³ | volume percent 35.78 | | | |
| positive control | | | | | | |
| Group IV: | 1300 | mg | | 4.84 | 3.84 | 4.81 |
| control | 2.18 | cm³ | | | | |

### Example 10

### Comparison of the Effects of Complex A on Mouse B 16 Melanoma in Peroral Administration and Intraabdominal Injection

The melanoma B 16 was implanted in 23 mice of the line C 57Bl by subcutaneous injection into the right thigh. The animals were divided into five groups of four to five mice each.

Group I received complex A at a dosage of 7 mg/kg as a suspension in olive oil which was administered perorally 3, 5, 7 and 9 days after transplantation of the tumor.

Group II was treated as group I, but using a dosage of 10 mg/kg administered on the 3rd, 4th, 5th, 6th, 7th, 8th, 9th and 10th day.

Group III was treated as group II, but using a dosage of 15 mg/kg.

Group IV received complex A 3, 5, 7 and 9 days after transplantation of the tumor at a dosage of 7 mg/kg, dissolved in DMSO, to which end the solution was administered by injection in the form of a 4% suspension in physiological saline.

Group V received no treatment.

On day 16 of the experiment the animals were sacrificed and examined.

The experimental results are summarized in Table 2. As can be seen, the anti-tumor effect is independent of the mode of administration. The tumor even disappeared completely in group 3.

TABLE 2

| | Average weight (mg) | |
|---|---|---|
| | and volume (cm³) | |
| Substance | of tumor | % inhibition |
| Group I: | 0.96 ± 0.14 | mass percent |
| complex A | p > 0.05 | 62.0 |
| (peroral) | 1.39 cm³ | volume percent |
| | | 84.1 |
| Group II: | 0.68 ± 0.34 | mass percent |
| complex A | 0.1 > p > 0.05 | 73.10 |
| (peroral) | 1.55 cm³ | volume percent |
| | | 76.0 |
| Group III: | 0 | mass percent |
| complex A | p < 0.05 | 100 |
| (peroral) | 0 | volume percent |
| | | 100 |
| Group IV: | 1.025 ± 0.39 | mass percent |
| complex A | p > 0.05 | 68.0 |
| (injection) | 1.5 cm³ | volume percent |
| | | 76.7 |
| Group V: | 2.525 ± 0.34 | — |
| control | 6.46 cm³ | |

### Example 11

### Effect of Complex A on Transplanted Colon Adenocarcinoma Tumor (AKATOL) in Intraabdominal Injection

A suspension of tumor cells was injected into the thighs of 15 mice of the BALB line. The animals received the complex A preparation at a dose of 5 mg/kg 4, 6, 8 and 11 days after implantation.

Group I received the preparation dissolved in DMSO and dispersed in a 4% suspension in physiological saline.

Group II received the preparation in Tween 60.

Group III remained untreated.

On day 21 of the experiment the animals were sacrificed and examined.

Table 3 shows the results. Administration in Tween 60 shows a significantly improved effect. Apparently, the inert detergent causes improved distribution of complex A in the organism.

TABLE 3

| | Average | | | |
|---|---|---|---|---|
| | volume of | Average | % Inhibition | |
| Group: | tumor (cm³) | weight (mg) | Mass percent | Volume percent |
| I | 1.315 | 1.25 | 60.4 | 87.1 |
| II | 0.217 | 0.25 | 95.85 | 97.8 |
| III | 10.164 | 6.02 | | |

### Example 12

### Effect of Complex A on Transplanted Colon Adenocarcinoma Tumor (AKATOL) in Peroral Administration

Tumor cells were transplanted in mice of the BALB line by injection into the thighs. 3 of 4 groups received the respective preparation on the peroral route 3, 4, 5, 6, 7, 8, 9 and 10 days after tumor implantation.

Group I: 10 mg/kg complex A suspended in olive oil.

Group II: 5 mg/kg complex A suspended in olive oil.

Group III: 10 mg/kg sarcolysine suspended in olive oil.

Group IV: no treatment.

On day 21 of the experiment the animals were sacrificed and examined. The results are summarized in Table 4. Complex A shows significantly increased effectiveness compared to sarcolysine (melphalan) in peroral administration as well.

TABLE 4

| | | Average weight of | |
|---|---|---|---|
| Substance | Dose mg/kg | tumor (g) | % inhibition |
| Group I: | 10 | 0.71 | 81.5 |
| complex A | | | |
| Group II: | 5 | 1.04 | 68 |
| complex A | | | |
| Group III: | 10 | 1.85 | 43 |
| sarcolysine | | | |
| positive | | | |
| control | | | |
| Group IV: | | 3.25 | |
| control | | | |

### Example 13

### Effect of Complex A on Ehrlich Ascites Adenocarcinoma

The tumor was implanted intraabdominally in white mice of no particular race.

Group I received complex A orally as a suspension in olive oil at a dosage of 10 mg/kg 3, 4, 5, 6, 7, 8, 9 and 10 days after tumor implantation.

Group II received the same treatment, but using sarcolysine instead of complex A.

Group III received no treatment.

The animals were sacrificed and examined on the 11^{th} day after tumor implantation.

The results are summarized in Table 5.

Complex A inhibits proliferation of tumor cells significantly better than sarcolysine and does not suppress the production of red bone marrow cells.

TABLE 5

| | Average | | | |
|---|---|---|---|---|
| | number | | | |
| | of tumor | | | |
| | cells | % | Average MI of | Average MI of |
| Substance | (10⁶) | inhibition | bone marrow, ‰ | tumor cells, ‰ |
| Group I: | 40′ | 75.3 | 0.95 ± 0.47 | 1.0 ± 0.7 |
| complex A | | | | |
| Group II: | 77 | 47.4 | 0.52 ± 0.26 | 2.0 ± 1.87 |
| sarcolysine | | | | |
| positive | | | | |
| control | | | | |
| Group III: | 162.4 | 1.3. | 1.0 ± 0.31 | 3.2 ± 0.44 |
| control | | | | |

### Example 14

### Influence of Complex A on the Formation of Nitrosyl Complexes of Hem Iron Using Walker Carcinosarcoma as Example

The oxygen metabolism in malignant cells is affected in such a way that increased NO production in these cells results in increased binding of NO to the hem iron, thereby disturbing the normal function thereof. The formation of such NO bonds becomes manifest in the EPR spectrum as a characteristic triplet overlying a singlet.

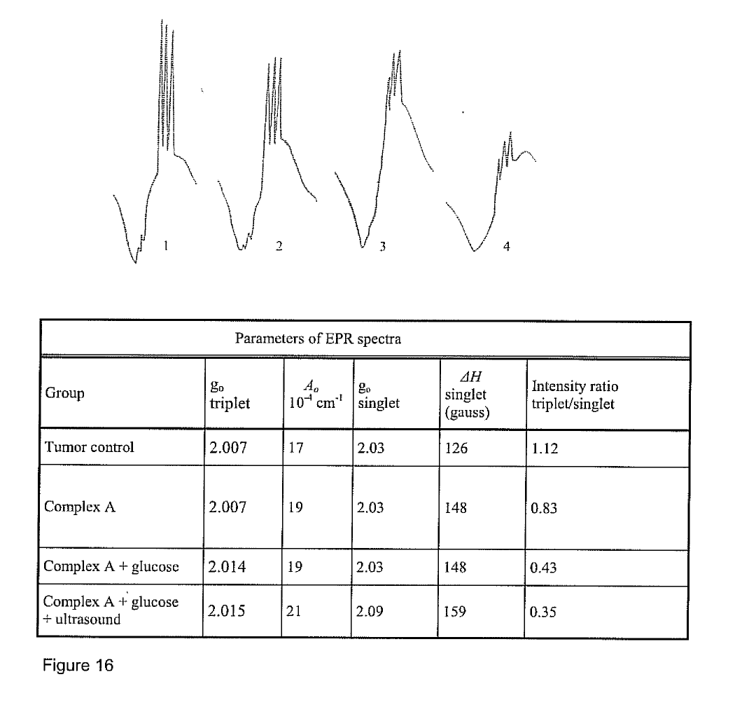

The effect of complex A on the concentration of Fe—NO bonds was investigated in an experiment using rats. The Walker carcinosarcoma was transplanted in the rats. 17 days after transplantation the animals were administered intraabdominally with complex A at a dosage of 30 mg/kg, suspended in liposomes from egg lecithin. Some of the animals also received glucose or glucose plus ultrasound. The results are summarized in FIG. 16. As can be seen, complex A, especially in combination with hyperglycemia and in particular hyperglycemia plus ultrasound, significantly reduces the concentration of Fe—NO bonds.

### Example 15

### Effect of Complex A on NADH and Calcium Homeostasis in Rats and on Thymocytes and Thymus in Ehrlich Ascites Carcinoma

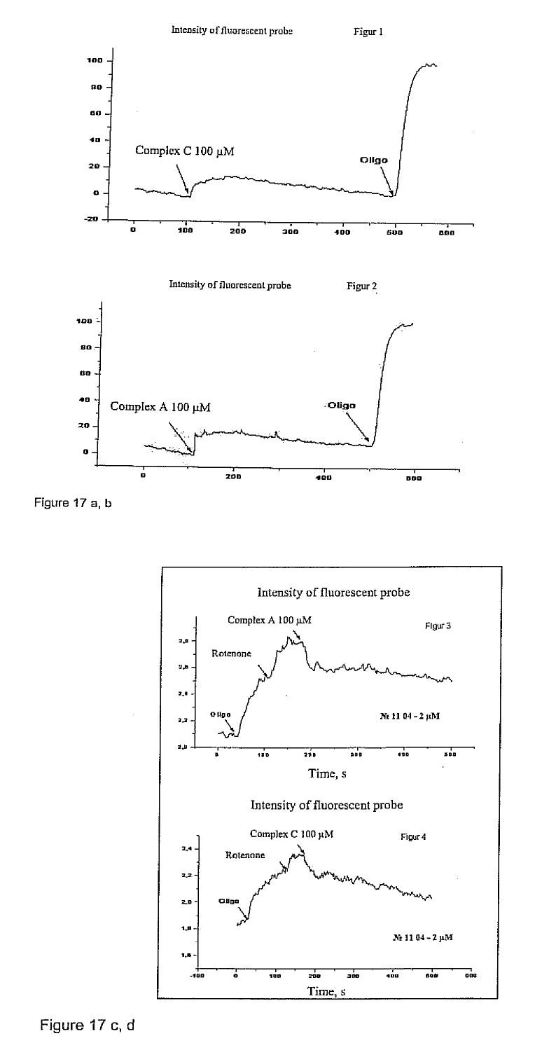

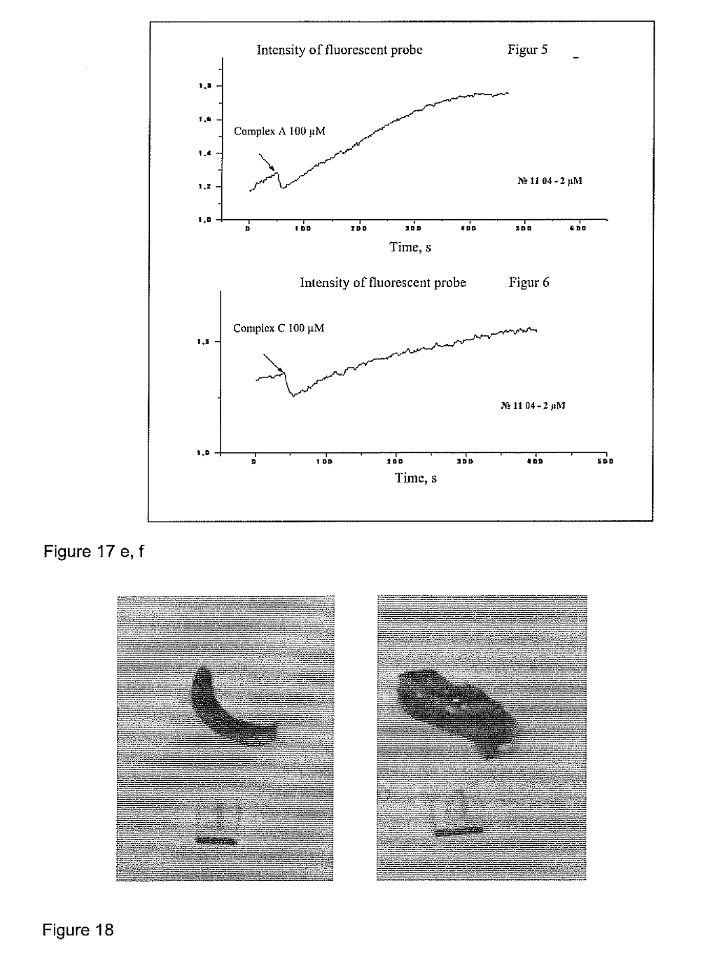

The Ehrlich ascites carcinoma (EAC) was obtained from white male mice of the strain MNR1 and the thymocytes from the thymus of rats of the Wistar line. Vitality of the cells was tested by vital staining using trypan blue and experiments and determined to be no less than 95% in all experiments. The NADH state in the EAC cells was assessed with reference to their fluorescence intensity. Measurement of the intracellular calcium concentration was performed according to the standard methodology of fluorescence using chlortetracycline. The membrane potential was measured using the fluorescent dye No. 1104. NADH is a source of electrons in the oxidative phosphorylation process. It was found that the complexes A and C have an effect on the NADH state and calcium concentration. They increase the amount of endogenous NADH to more than 20% and reduce the response to oligomycin (FIG. 17a, 17b). These effects are directed to inhibiting the NAD-dependent dehydrogenase. As a result, the respiratory chain in the tumor cells is inhibited.

Furthermore, it was possible to demonstrate that the complexes A and C, depending on the dose, increase the concentration of cytosolic calcium in EAC cells and thymocytes. The concentration of intracellular calcium is a trigger for a number of intracellular processes (FIG. 17c, 17d).

Moreover, the complexes A and C have an influence on the membrane potential of thymocytes. It was found that hyperpolarization of the cell membranes (FIG. 17e, 17f) induces opening of calcium channels and depolarization of the mitochondrial membranes.

### Example 16

### Radioprotective Effect of Complex A and Complex C

White mice of no particular race were irradiated with a sublethal dose of 6 Gray. Two hours prior to irradiation and three days after irradiation part of the mice were treated with complex A and complex C at a dose of 5 mg/kg each time. The animals were sacrificed on day 9 after irradiation, and the number of hematopoietic cells in the spleen was determined. No hematopoietic cells were detected in the spleen of untreated animals. The treated animals had a normal number of about 40 colonies of hematopoietic cells.

FIG. 18 shows a comparison of the spleen of an untreated mouse and the spleen of a treated mouse.

### Example 17

### Antioxidant Properties of Complex A and Complex B

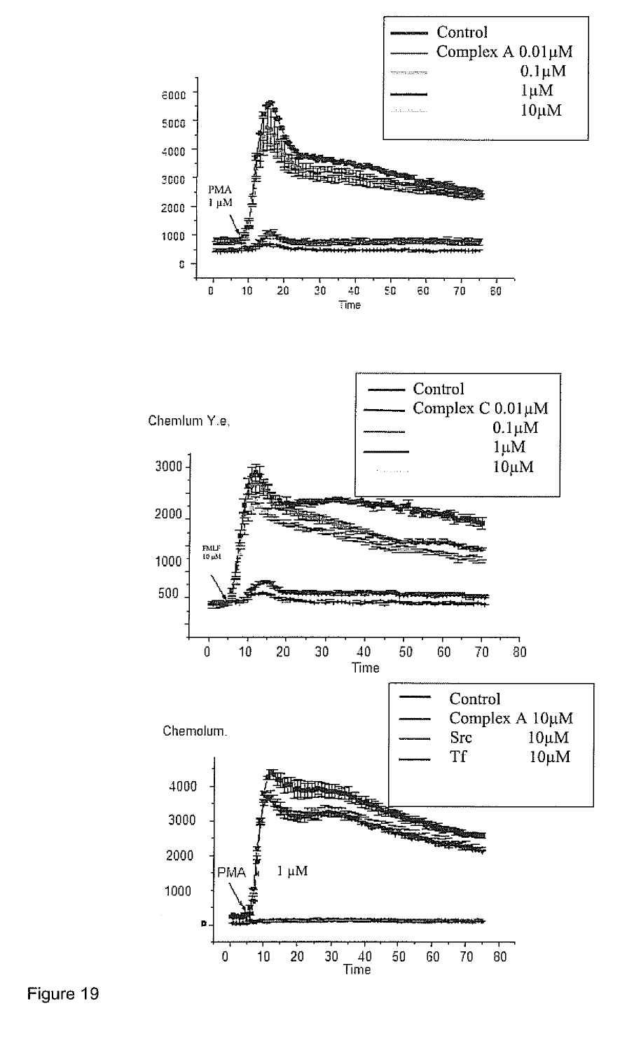

The complexes A and C show a pronounced effect as antioxidant in an environment having an excess of reactive oxygen species (ROS). Thus, ROS formation in neutrophils obtained from male mice of the NMRI line was triggered by two activators: phorbol 12-myristate 13-acetate (PMA) and N-formyl-methionyl-leucylphenylalanine (fMLF). As shown in FIGS. 19a and b, addition of complex A and complex C, depending on the dosage, can reduce or completely suppress formation of ROS in neutrophils. FIG. 19c shows that this effect is hardly achieved when using sarcolysine and tegafur.

### Example 18

### Influence of Complexes A and C on the Permeability of the Mitochondrial Membrane in Liver Cells of Rats Compared to Sarcolysine

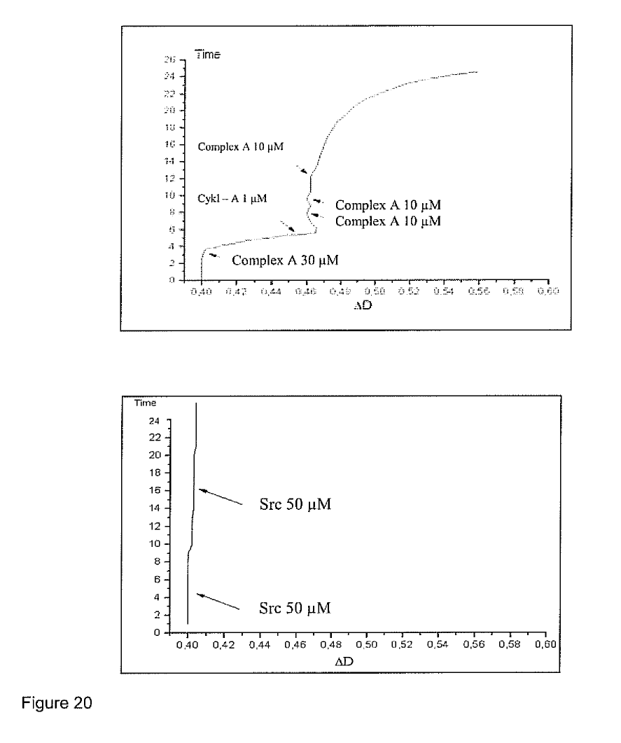

The optical density of the cell suspension was measured as an indicator of permeability. It was found that the complexes A and C have a strong influence on the permeability, which is stopped when adding cyclosporin A, but restored upon further addition of the complexes (FIG. 20a). FIG. 20b shows that sarcolysine does not have such influence.

It was determined that the complexes A and C have a strong influence on the permeability of mitochondrial membranes. They open the cyclosporin A sensor pores up to high permeability (up to 1500 Daltons). They irreversibly reduce the membrane potential. They give rise to osmotic swelling of the mitochondrial matrix. They contribute to the destruction of the mitochondrial membrane. Apparently, the main factor of this effect is copper as central atom of the complex. It not only changes the properties of sarcolysine and tegafur, but apparently develops biological effects by itself.

### Example 19

### Effect of Complex A on Hep-2 Tumor Cells of Human Laryngeal Carcinoma

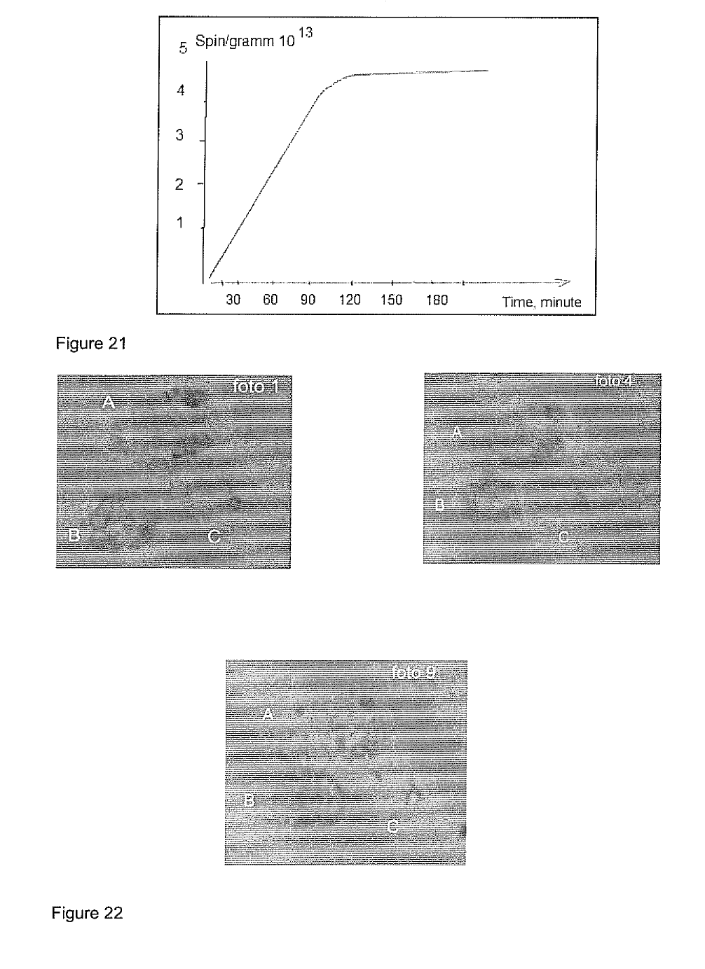

Complex A dissolved in Tween 80 was mixed with liposomes from lecithin. This mixture was added with a culture of Hep-2 cells, and the EPR spectra were recorded over time. A culture having no complex A preparation was measured for comparison. The EPR spectra allow quantitative determination of the copper content in the tumor cells. It was found that the Hep-2 cells absorb copper in an amount of 4.5×10¹³ spin per gram. This corresponds to an intake of 10 copper-containing molecules in a cell. This saturation value of the cells is reached within 1.5 to 2 hours (FIG. 21). In addition, the cell suspension was purified by filtration, and the filtrate containing the cellular components was fractionated using a centrifuge. It was found that the copper complex had accumulated almost entirely in the nuclear chromatin, as determined by measuring the EPR signal of copper.

### Example 20

### In Vitro Effect of Complex A on Human Mammary Gland Tumor Cells

From a female patient with mammary carcinoma already showing signs of decay, postoperational material was collected and the tumor cells were separated therefrom. A suspension of these tumor cells was added with trypan blue dye and complex A. During the course of the test, three cells situated closely to each other (A, B and C) were documented by photography (FIGS. 22a, 22b and 22c). After incubating for 18 minutes, wrinkling of the membrane of cell A and invagination of the membrane of cell B can be recognized. After 28 minutes of incubation, cell fragments become stained with trypan blue, a sign that apoptosis has begun. After 43 minutes of incubation, membrane components and the contents thereof detach from the cells.

### Example 21

### In Vitro Effect of Complexes A and B on Human Mammary Gland Tumor Cells in Comparison to the Effect of Sarcolysine (Src) and an Untreated Control

Tumor cells were collected from the surgical material of female patients suffering from mammary carcinoma at two different stages. The tumor tissue was added with sterile 0.25% trypsin solution and centrifuged repeatedly in RPMI-1640 nutrient medium for 15 minutes at 1500 revolutions per minute. The amount of resulting cells was counted and the suspension of tumor cells divided into five equal groups. The RPMI-1640 nutrient medium was added with 12% fetal calf serum. The cells were incubated for 48 h at 37° C. in a 5% CO₂ stream.

The preparations were dissolved in 4% DMSO and physiological saline and acidified with HCl.

Two experiments were performed using cancer cells derived from tumors of different stages. To differentiate the various stages of cancer, the TNM classification is used internationally which takes into account the tumor size (T), lymph node affection (N=nodal status) and the extent of metastasis (M).

### Experiment 1: T₂N₁M₀ Stage

(T₂: tumor up to 5 cm; N₁; metastases in 3 lymph nodes; M₀: no distant metastases). A suspension including 40 million cells was added with 50 μl of each preparation.

### Experiment 2: T₄N₂M₀ Stage

(T₃: large tumor spreading into the tissue surrounding the breast; N₂: metastases up to 9 axillary lymph nodes, M₀: no distant metastases). A suspension including 40 million cells was added with 50 μl of each preparation.

The 5 groups with cancer cells received the following:

Group I: Scr

Group II: complex B

Group III: complex A

Group IV: control

After subjecting the preparations to flow incubation for 60 minutes, trypan blue stained cells were produced, and dead cells and apoptosis could be determined; dead cells and apoptotic cells were determined in specific units of time and morphological findings.

### Experiment 1

| Group: | Dead cells, % | Living cells, % |
|---|---|---|
| I Src | 46.0 ± 2.22 | 54.0 ± 2.22 |
| II complex B | 67.0 ± 2.14 | 33.0 ± 2.10 |
| III complex A | 76.0 ± 2.06 | 24.0 ± 1.90 |
| IV control | 18.0 ± 1.71 | 82.0 ± 1.71 |

### Experiment 2

| Group: | Dead cells, % | Living cells, % |
|---|---|---|
| I Src | 49.0 ± 2.23 | 51.0 ± 2.23 |
| II complex B | 73.0 ± 2.02 | 27.0 ± 1.98 |
| III complex A | 82.0 ± 1.78 | 18.0 ± 1.71 |
| IV control | 13.0 ± 1.50 | 87.0 ± 1.50 |

1. A copper-organic complex obtained by reacting a copper(II) acylate with an organic compound selected from 4-[bis(2-chloroethyl)amino]-D,L-phenylalanine (sarcolysine), the hydrochloride thereof, N-(2-furanidil)-5-fluorouracil (tegafur) and aminocarbonylaziridine (leacadin) at acidic to neutral pH in a chloroform/methanol mixture.

2. The copper-organic complex according to claim 1, wherein copper(II) acetate or copper(II) propionate is used as copper(II) acylate.

3. The copper-organic complex according to claim 1, wherein the chloroform/methanol mixture is used at a ratio of from 1:1 to 3:1.

4. The copper-organic complex according to claim 1, wherein it is a copper-sarcolysine hydrochloride complex with a melting point of 147° C., which complex comprises Cu: 15.0%, C, 37.3% H, 4.43%, Cl: 24.4%, N: 6.74% and oxygen and is prepared by reacting sarcolysine hydrochloride and copper(II) acetate at a pH of about 2 to about 3, the deviations of the quoted analytical values for carbon, nitrogen and chlorine being less than 3% on average, and the deviations of the quoted analytical values for hydrogen and copper being up to 4.5% on average.

5. The copper-organic complex according to claim 1, wherein it is a copper-sarcolysine complex with a melting point of 177° C., which complex comprises Cu: 9.2%, C, 45.3%, H, 5.4%, Cl: 20.6%, N: 8.1% and oxygen and is prepared by reacting sarcolysine and copper(II) acetate at a pH of about 6 to about 7, the deviations of the quoted analytical values for carbon, nitrogen and chlorine being less than 3% on average, and the deviations of the quoted analytical values for hydrogen and copper being up to 4.5% on average.

6. The copper-organic complex according to claim 1, wherein it is a copper-tegafur complex with a melting point of 127° C., which complex comprises 26.8% Cu, 18.16% carbon, 3.08% hydrogen, 25.3% chlorine, 3.39% nitrogen, oxygen and fluorine and is prepared by reacting tegafur and copper(II) acetate at a pH of about 1, the deviations of the quoted analytical values for carbon, nitrogen and chlorine being less than 3% on average, and the deviations of the quoted analytical values for hydrogen and copper being up to 4.5% on average.

7. A pharmaceutical composition comprising at least one copper-organic complex according to claim 1 and optionally pharmaceutical adjuvants and/or excipients.

8. (canceled)

9. (canceled)

10. (canceled)

11. (canceled)

12. (canceled)

13. A method of preparing a copper-organic complex, wherein copper(II) acylate is reacted with an organic compound selected from 4-[bis(2-chloroethyl)amino]-D,L-phenylalanine (sarcolysine), the hydrochloride thereof, N-(2-furanidil)-5-fluorouracil (tegafur) and aminocarbonylaziridine (leacadin) at acidic to neutral pH in a chloroform/methanol mixture.

14. The method according to claim 13, wherein the copper(II) acylate and the organic compound selected from 4-[bis(2-chloroethyl)amino]-D,L-phenylalanine (sarcolysine), the hydrochloride thereof, N-(2-furanidil)-5-fluorouracil (tegafur) and aminocarbonylaziridine (leacadin) are each dissolved in a chloroform/methanol mixture, combined, acidified, if necessary, and heated to temperatures of at least 50° C., and the precipitate resulting after cooling is washed, if necessary, and dried.

15. The method of claim 13, wherein the chloroform/methanol mixture is used at a ratio of from 1:1 to 3:1.
